(19) United States
(12) Patent Application Publication
(10) Pub. No.: US 2026/0224653 A1
(43) Pub. Date: Aug. 6, 2026

(54) METHOD OF TREATING RAYNAUD'S CONDITIONS OR ERECTILE DYSFUNCTION

(71) Applicant: Bioharvest Ltd., Rehovot (IL)

(72) Inventors: Yoheved HAGAY, Rehovot (IL); Malkit AZACHI, Rehovot (IL); Ilan SOBEL, Raanana (IL)

(21) Appl. No.: 19/155,014

(22) PCT Filed: Feb. 6, 2024
(86) PCT No.: PCT/IL2024/050137

§ 371 Date: Aug. 8, 2025

Related U.S. Application Data
(60) Provisional application No. 63/444,264, filed on Feb. 9, 2023.
(60) Provisional application No. 63/457,220, filed on Apr. 5, 2023.

(51) Int. Cl. A61K 36/87; A61K 9/00; A61P 9/08; A61P 15/10

(52) U.S. Cl. CPC A61K 36/87; A61K 9/0014; A61P 9/08; A61P 15/10

(57) ABSTRACT

The invention provides method for treating Raynaud's conditions such as Raynaud's syndrome, Raynaud's phenomenon, and Raynaud's disease by administering to a subject in need a pharmaceutical or a dietary supplement comprising grape cell culture grown in vitro. Further provided method for treating erectile dysfunction by administering to a subject in need a pharmaceutical or a dietary supplement comprising grape cell culture grown in vitro. For example, red grape cell culture grown in vitro in bioreactors are administered to treat or reduce the severity of a symptom of a Raynaud's condition and/or to ameliorate erectile dysfunction.

### BACKGROUND OF THE INVENTION

Raynaud's conditions (e.g., Raynaud's syndrome, Raynaud's phenomenon, and Raynaud's disease) are circulatory conditions characterized by over sensitivity to cold. Symptoms of Raynaud's conditions include:

Cold fingers or toes.

Areas of skin that turn white then blue.

Numb, prickly feeling or stinging pain upon warming or stress relief.

Raynaud's most commonly affects fingers and toes. But it also can affect other areas of the body, such as nose, lips, ears and even nipples.

Erectile dysfunction (ED) is defined as a persistent inability to obtain and/or maintain appropriate penile rigidity to allow a satisfactory sexual intercourse, and it is responsible for a deep and significant compromise in the quality of life of men and their partners.

### SUMMARY OF THE INVENTION

In some embodiments, there is provided a method of treating Raynaud's condition in a subject of need thereof comprising administering to the subject a composition comprising a therapeutically effective amount of red grape cell culture grown in vitro. In some embodiments, there is provided a method of treating Raynaud's condition in a subject of need thereof comprising administering to the subject a composition comprising a therapeutically effective amount of red grape cell culture grown in vitro and a pharmaceutically or dietary acceptable carrier. In some embodiments, the Raynaud's condition is Raynaud's syndrome, Raynaud's phenomenon, and Raynaud's disease.

In some embodiments, there is provided a method of treating a subject with erectile dysfunction, the method comprising the step of administering to the subject a composition comprising a therapeutically effective amount of red grape cell culture grown in vitro.

In some embodiments, there is provided a method of treating a subject with erectile dysfunction, said method comprising the step of administering to said subject a composition comprising a therapeutically effective amount of red grape cell culture grown in vitro and a pharmaceutically acceptable carrier or dietary acceptable carrier.

In some embodiments, there is provided a composition comprising a therapeutically effective amount of red grape cell culture grown in vitro for treating Raynaud's condition in a subject of need thereof. In some embodiments, there is provided a composition comprising a therapeutically effective amount of red grape cell culture grown in vitro and a pharmaceutically or dietary acceptable carrier for treating Raynaud's condition in a subject of need thereof.

In some embodiments, there is provided a composition comprising a therapeutically effective amount of red grape cell culture grown in vitro for treating a subject with erectile dysfunction. In some embodiments, there is provided a composition comprising a therapeutically effective amount of red grape cell culture grown in vitro and a pharmaceutically or dietary acceptable carrier for treating a subject with erectile dysfunction.

In some embodiments, the composition is an oral composition in a form of a capsule consisting of red grape cell culture grown in vitro.

In some embodiments, the capsule comprises 100-800, 100-700, 100-600, 100-500, 200-400, 200-700, 200-600, 200-500 or 200-400 mg red grape cells grown in vitro dry powder.

In some embodiments, the capsule comprises about 100, 200, 200, 400, 500 or 600 mg red grape cells grown in vitro dry powder.

In some embodiments, the capsule comprises about 100, 200, 200, 400, 500 or 600 mg red grape cells grown in vitro dry powder that contains between 20-70, 20-60, 20-50 or 20-40 mg polyphenols.

In some embodiments, the capsule comprises about 100, 200, 200, 400, 500 or 600 mg red grape cells grown in vitro dry powder that contains between 10-60, 10-50 or 10-40 mg polyphenols.

In some embodiments, the capsule comprises about 100, 200, 200, 400, 500 or 600 mg red grape cells grown in vitro dry powder that contains between 2-12, 3-10, 4-8 or 5-7 mg polyphenols.

In some embodiments, the capsule comprises about 100, 200, 200, 400, 500 or 600 mg red grape cells grown in vitro dry powder that contains about 2, 3, 4, 5, 6, 7, 8, 9 or 10 mg or more piceid resveratrol.

In some embodiments, the capsule comprises about 100, 200, 200, 400, 500 or 600 mg red grape cells grown in vitro dry powder that contains about 6 mg piceid resveratrol.

In some embodiments, the capsule comprises about 400 mg red grape cells dry powder which contains about 6 mg piceid resveratrol and 20-40 mg polyphenols.

In some embodiments, the red grape cell culture grown in vitro are grown in bioreactors.

In some embodiments, wherein the red grape cell culture grown in vitro are in a form of a powder comprising a cell line culture of grape cells grown in vitro in a large scale up process, whereby the cell line culture of grape cells is derived from one or more of grape-berry cross section, grape-berry skin, grape-berry flesh, grape seed, grape embryo of seeded or seedless cultivars or grape seed coat; wherein the cell line culture of grape cells includes resveratrol in an amount of at least 1000 mg/kg powder. In some embodiments, the composition in the capsule is Vinia®.

In some embodiments, there is provided a method of treating Raynaud's condition in a subject of need thereof comprising locally administering to the subject a topical composition comprising a therapeutically effective amount of red grape cell culture grown in vitro and a pharmaceutically or dermatologically acceptable carrier.

In some embodiments, there is provided a method of treating erectile dysfunction in a subject of need thereof comprising locally administering to the subject a topical composition comprising a therapeutically effective amount of red grape cell culture grown in vitro and a pharmaceutically or dermatologically acceptable carrier.

In some embodiments, there is provided a topical composition comprising a therapeutically effective amount of red grape cell culture grown in vitro and a pharmaceutically or dermatologically acceptable carrier for locally treating Raynaud's condition.

In some embodiments, there is provided a topical composition comprising a therapeutically effective amount of red grape cell culture grown in vitro and a pharmaceutically or dermatologically acceptable carrier for locally treating erectile dysfunction.

### DETAILED DESCRIPTION OF THE EMBODIMENTS OF THE INVENTION

The invention provides a composition and method for orally and/or topically treating Raynaud's conditions such as Raynaud's syndrome, Raynaud's phenomenon, and Raynaud's disease by administering to a subject in need a pharmaceutical or a dietary supplement comprising grape cell culture grown in vitro.

In some embodiments, a topical composition comprising grape cell culture grown in vitro is locally administered to the subject to treat the Raynaud's condition symptoms. In some embodiments, the treatment is a combined treatment by both orally administered dosage form and locally administering a topical dosage form.

Further provided method for orally or topically treating erectile dysfunction by administering to a subject in need a pharmaceutical or a dietary supplement comprising grape cell culture grown in vitro. For example, red grape cell culture grown in vitro in bioreactors are administered to treat or reduce the severity of a symptom of a Raynaud's condition and/or to ameliorate erectile dysfunction. In some embodiments, a topical composition comprising grape cell culture grown in vitro is locally administered to the subject to treat the erectile dysfunction. In some embodiments, the treatment is a combined treatment by both orally administered dosage form and locally administering a topical dosage form.

### Definitions

As used herein, the terms “about” and “approximately” are used interchangeably, and when used to refer to modify a numerical value, encompass a range of uncertainty of the numerical value of from 0% to 10% of the numerical value.

As used herein, the singular forms “a,” “an,” and “the” include plural referents unless the context clearly dictates otherwise.

The terms “treat,” “treating,” and “treatment,” in the context of treating a disease, disorder, or condition, are meant to include alleviating a disorder, disease, or condition, or one or more of the symptoms associated with the disorder, disease, or condition; or to slow the progression of a disease, disorder or condition or of one or more symptoms thereof.

As used herein, the terms “subject” “individual,” or “patient,” are used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the disease or disorder is associated with dysregulation of blood flow and sympathetic nervous system overactivity. In some embodiments, the disease or disorder is characterized by neuropathic pain, vasoconstriction, burning pain, body temperature changes of the subject, hyperesthesia, changes in skin or tissue color, edema, changes in skin turgor, ruble, pallor, cyanosis, vasospasm, or any combination thereof.

Raynaud's conditions (e.g., Raynaud's syndrome, Raynaud's phenomenon, and Raynaud's disease) are circulatory conditions characterized by over sensitivity to cold.

Symptoms of Raynaud's conditions include:

Cold fingers or toes.

Areas of skin that turn white then blue.

Numb, prickly feeling or stinging pain upon warming or stress relief.

Raynaud's most commonly affects fingers and toes. But it also can affect other areas of the body, such as nose, lips, ears and even nipples.

There are two main types of the condition.
Primary Raynaud's. Also called Raynaud's disease, this most common form isn't the result of another medical condition. It can be so mild that many people with primary Raynaud's don't seek treatment.
Secondary Raynaud's. Also called Raynaud's phenomenon, this form develops because of another health condition. Although secondary Raynaud's is less common than the primary form, it tends to be more serious. Symptoms of secondary Raynaud's usually appear around age 40.

In some embodiments, there is provided a method of treating Raynaud's condition in a subject of need thereof comprising administering to the subject a therapeutically effective amount of a composition comprising red grape cell culture grown in vitro.

In some embodiments, there is provided a method of treating Raynaud's condition in a subject of need thereof comprising administering to the subject a therapeutically effective amount of a composition comprising red grape cell culture grown in vitro and a pharmaceutically acceptable carrier.

In some embodiments, the Raynaud's condition is Raynaud's syndrome, Raynaud's phenomenon, and Raynaud's disease. According to some embodiments, the red grape cell culture grown in vitro are grown in bioreactors.

The term “therapeutically effective amount” or “effective amount” as used herein in relation to Raynaud's condition means that the amount of the grape cells grown in vitro contained in the composition administered to the subject in need is of sufficient quantity to achieve the intended purpose, such as, in this case, to reduce cold fingers or toes, nose, lips, ears or nipples, changes in skin color and to reduce or eliminate numb, prickly feeling or stinging pain upon warming or stress relief.

In some embodiments, the red grape cell culture grown in vitro are grown in bioreactors are in a form of a powder comprising a cell line culture of grape cells grown in vitro in a large scale up process, whereby the cell line culture of grape cells is derived from one or more of grape-berry cross section, grape-berry skin, grape-berry flesh, grape seed, grape embryo of seeded or seedless cultivars or grape seed coat; wherein the cell line culture of grape cells includes resveratrol in an amount of at least 1000 mg/kg powder.

In some embodiments, the composition is Vinia® or comprises Vinia®.

In some embodiments, the composition is in a form of a capsule comprising red grape cells grown in vitro dry powder.

In some embodiments, the capsule comprises 100-800, 100-700, 100-600, 100-500, 200-400, 200-700, 200-600, 200-500 or 200-400 mg red grape cells grown in vitro dry powder.

In some embodiments, the capsule comprises about 100, 200, 200, 400, 500 or 600 mg red grape cells grown in vitro dry powder.

In some embodiments, the capsule comprises about 100, 200, 200, 400, 500 or 600 mg red grape cells grown in vitro dry powder that contains between 20-70, 20-60, 20-50 or 20-40 mg polyphenols.

In some embodiments, the capsule comprises about 100, 200, 200, 400, 500 or 600 mg red grape cells grown in vitro dry powder that contains between 10-60, 10-50 or 10-40 mg polyphenols.

In some embodiments, the capsule comprises about 100, 200, 200, 400, 500 or 600 mg red grape cells grown in vitro dry powder that contains between 2-12, 3-10, 4-8 or 5-7 mg polyphenols.

In some embodiments, the capsule comprises about 100, 200, 200, 400, 500 or 600 mg red grape cells grown in vitro dry powder that contains about 2, 3, 4, 5, 6, 7, 8, 9 or 10 or more mg piceid resveratrol.

In some embodiments, the capsule comprises about 100, 200, 200, 400, 500 or 600 mg red grape cells grown in vitro dry powder that contains about 6 mg piceid resveratrol.

In some embodiments, the capsule comprises about 400 mg red grape cells dry powder which contains about 6 mg piceid resveratrol and 20-40 mg polyphenols.

In some embodiments, there is provided a method of treating Raynaud's condition in a subject of need thereof comprising locally administering to the subject a topical composition comprising a therapeutically effective amount of red grape cell culture grown in vitro and a pharmaceutically or dermatologically acceptable carrier.

In some embodiments, there is provided a topical composition comprising a therapeutically effective amount of red grape cell culture grown in vitro and a pharmaceutically or dermatologically acceptable carrier for locally treating Raynaud's condition.

Compositions of the present invention may include a carrier. Depending on the kind of compositions of the present invention, a carrier may be a dietary suitable carrier or a pharmaceutically acceptable carrier, as long as it is compatible with the particular kind of compositions of the present invention. Examples of a dietary suitable carrier include, but are not limited to, dietary suitable excipients, diluents, and carriers. Examples of a pharmaceutically acceptable carrier include, but are not limited to, biocompatible vehicles, adjuvants, additives, and diluents to achieve a composition usable as a dosage form.

The compositions comprising grape cell line grown in vitro may be used alone or in combination with other biologically active ingredients. The composition of the invention, alone or in combination with other active ingredients, may be administered to a subject in a single dose or multiple doses over a period of time, generally by oral administration. Various administration patterns will be apparent to those skilled in the art. The dosage ranges for the administration of the compositions of the invention are those large enough to produce the desired effect. The dosage should not be so large as to cause any adverse side effects, such as unwanted cross-reactions and the like. Generally, the dosage will vary with the age, weight, sex, condition, and extent of a condition in a subject, and the intended purpose. The dosage can be determined by one of skill in the art without undue experimentation. The dosage can be adjusted in the event of any counter indications, tolerance, or similar conditions. Those of skill in the art can readily evaluate such factors and, based on this information, determine the particular effective concentration of a composition of the present invention to be used for an intended purpose.

In some embodiments, the pharmaceutical topical composition as defined above that comprises red grape cell culture grown in vitro further comprises at least one ingredient selected from the group consisting of: glyceryl stearate & PEG-100 stearate, acetyl alcohol, allantoin, petrolatum, steareth-21, tocopheryl acetate, xanthan gum, dipotassium glycyrrhizate, triethanolamine, bisabolol, disodium EDTA, vitamin B3, anti-irritation agent, anti-oxidant, terpene, cannabis terpene, anti-skin redness agent, anti-adherent, binder, coating, disintegrant, flavour, colour, lubricant, glidant, sorbent, preservative, filler, emulsifier, humectant, thickener, skin nourishing skin moistening agent, occlusive agent, emollient agent, calming agent, natural smell agent, suspending agent, soothing agent, pH adjustment agent, purified water, shea butter, lavender oil, and any combination thereof.

In some embodiments, the topical composition comprising red grape cell line grown in vitro is in a form of a cream, soap, cleansing agent, lotion, moisturizer, ointment, lotion, gel, foam, solution, or powder. The grape cell line grown in vitro is typically added to a topical composition in an amount of between 1-10%, 2-8%, 2-7%, 2-6%, 2-5%, 2-4%, 1-4%, 1-6% or 1-8% of a powder that is formed as explained for example in WO 2014/068557. The powder of the red grape cell line grown in vitro in some embodiments comprises 1000 mg/kg or more resveratrol.

In some embodiments, the fruit cell culture grown in vitro is red grape cells (RGC or Erectile dysfunction (ED) is defined as a persistent inability to obtain and/or maintain appropriate penile rigidity to allow a satisfactory sexual intercourse, and it is responsible for a deep and significant compromise in the quality of life of men and their partners. Reasons for erectile dysfunctions are many and include, but are not limited to psychological factors, endocrine factors, neurogenic factors (e.g. neural trauma, spinal cord injury), vascular factors, age factors (being common at old age), disease conditions (e.g. cardiovascular diseases, hypertension, diabetes) and external factors such as passive and active smoking. In addition, sometimes ED is a side effect upon treatment with many drugs such as antidepressants, antipsychotics and certain antihypertensives. Erectile dysfunction is commonly also referred to as impotence.

The term “therapeutically effective amount” or “effective amount” as used herein in relation to ED means that the amount of the grape cells grown in vitro contained in the composition administered to the subject in need is of sufficient quantity to achieve the intended purpose, such as, in this case, as a tool for reduced time to erection, increased length of erection and/or increased sexual desire.

In some embodiments, there is provided a method of treating erectile dysfunction in a subject of need thereof comprising administering to the subject a therapeutically effective amount of a composition comprising red grape cell culture grown in vitro.

In some embodiments, there is provided a method of treating erectile dysfunction in a subject of need thereof comprising administering to the subject a therapeutically effective amount of a composition comprising red grape cell culture grown in vitro and a pharmaceutically acceptable carrier.

According to some embodiments, the red grape cell culture grown in vitro are grown in bioreactors.

In some embodiments, the composition is in a form of a powder comprising a cell line culture of grape cells grown in vitro in a large scale up process, whereby the cell line culture of grape cells is derived from one or more of grape-berry cross section, grape-berry skin, grape-berry flesh, grape seed, grape embryo of seeded or seedless cultivars or grape seed coat; wherein the cell line culture of grape cells includes resveratrol in an amount of at least 1000 mg/kg powder.

In some embodiments, the composition is Vinia® or comprises Vinia®.

In some embodiments, the composition is in a form of a capsule comprising red grape cells grown in vitro dry powder.

In some embodiments, the capsule comprises 100-800, 100-700, 100-600, 100-500, 200-400, 200-700, 200-600, 200-500 or 200-400 mg red grape cells grown in vitro dry powder.

In some embodiments, the capsule comprises about 100, 200, 200, 400, 500 or 600 mg red grape cells grown in vitro dry powder.

In some embodiments, the capsule comprises about 100, 200, 200, 400, 500 or 600 mg red grape cells grown in vitro dry powder that contains between 20-70, 20-60, 20-50 or 20-40 mg polyphenols.

In some embodiments, the capsule comprises about 100, 200, 200, 400, 500 or 600 mg red grape cells grown in vitro dry powder that contains between 10-60, 10-50 or 10-40 mg polyphenols.

In some embodiments, the capsule comprises about 100, 200, 200, 400, 500 or 600 mg red grape cells grown in vitro drsy powder that contains between 2-12, 3-10, 4-8 or 5-7 mg piceid resveratrol.

In some embodiments, the capsule comprises about 100, 200, 200, 400, 500 or 600 mg red grape cells grown in vitro dry powder that contains about 2, 3, 4, 5, 6, 7, 8, 9 or 10 mg piceid resveratrol.

In some embodiments, the capsule comprises about 100, 200, 200, 400, 500 or 600 mg red grape cells grown in vitro dry powder that contains about 6 mg piceid resveratrol.

In some embodiments, the capsule comprises about 400 mg red grape cells dry powder which contains about 6 mg piceid resveratrol and 20-40 mg polyphenols.

Compositions of the invention may include a carrier. Depending on the kind of compositions of the present invention, a carrier may be a dietary suitable carrier or a pharmaceutically acceptable carrier, as long as it is compatible with the particular kind of compositions of the present invention. Examples of a dietary suitable carrier include, but are not limited to, dietary suitable excipients, diluents, and carriers. Examples of a pharmaceutically acceptable carrier include, but are not limited to, biocompatible vehicles, adjuvants, additives, and diluents to achieve a composition usable as a dosage form. As used herein, the terms “pharmaceutically acceptable,” “physiologically tolerable,” and grammatical variations thereof, as they refer to compositions, carriers, diluents, and reagents, are used interchangeably and represent that the materials are capable of administration to or upon a mammal without the production of undesirable physiological effects.

The compositions comprising grape cell line grown in vitro may be used alone or in combination with other biologically active ingredients. The composition of the invention, alone or in combination with other active ingredients, may be administered to a subject in a single dose or multiple doses over a period of time, generally by oral administration. Various administration patterns will be apparent to those skilled in the art. The dosage ranges for the administration of the compositions of the invention are those large enough to produce the desired effect. The dosage should not be so large as to cause any adverse side effects, such as unwanted cross-reactions and the like. Generally, the dosage will vary with the age, weight, sex, condition, and extent of a condition in a subject, and the intended purpose. The dosage can be determined by one of skill in the art without undue experimentation. The dosage can be adjusted in the event of any counter indications, tolerance, or similar conditions. Those of skill in the art can readily evaluate such factors and, based on this information, determine the particular effective concentration of a composition of the present invention to be used for an intended purpose.

In some embodiments, the fruit cell culture grown in vitro is in vitro grown red grape cells (RGC or RGC product). RGC powder contains a matrix of polyphenols including piceid resveratrol, quercetin, anthocyanins, and tannins. In some embodiments, the RGC powder is composition in a form of a powder comprising a cell line culture of grape cells grown in vitro in a large scale up process, whereby the cell line culture of grape cells is derived from one or more of grape-berry cross section, grape-berry skin, grape-berry flesh, grape seed, grape embryo of seeded or seedless cultivars or grape seed coat; wherein the cell line culture of grape cells includes resveratrol in an amount of at least 1000 mg/kg powder. In some embodiments, the RGC is prepared as detailed in WO 2014/068557. In some embodiments, the RGC powder is equivalent to VINIA®.

Accordingly, VINIA® is a source of the whole matrix of nutrients and polyphenols found throughout the red grape and in red wine in their natural state (physicochemical state) without any genetic manipulation and contains polyphenols (piceid resveratrol, tannins, quercetin, catechins, anthocyanins), vitamins, protein, fiber. Its advantages are in the absence of solvent residuals and the typical taste without bitterness and astringent.

In some embodiments, there is provided a method of treating erectile dysfunction in a subject of need thereof comprising locally administering to the subject a topical composition comprising a therapeutically effective amount of red grape cell culture grown in vitro and a pharmaceutically or dermatologically acceptable carrier.

In some embodiments, there is provided a topical composition comprising a therapeutically effective amount of red grape cell culture grown in vitro and a pharmaceutically or dermatologically acceptable carrier for locally treating erectile dysfunction.

### EXAMPLES

The following examples further illustrate the embodiments disclosed herein. These examples are provided only for purpose of illustrating the preferred embodiments of the invention and do not limit the invention in any manner

### Example 1: Effect of Vinia® on Raynaud's Conditions

Vinia® has been on the market since 2018. Some consumers which purchased the Vinia® product sent their feedback to the company asserting that Vinia® has beneficial effects on their cold hands and/or feet symptoms.

Questionnaire were received from 10 subjects who had experienced a positive effect. 90% of the respondents took one capsule of Vinia@ daily. 10% of the respondents took two capsules of Vinia® daily. All of the subjects took Vinia® for more than 3 months. 90% of respondents took Vinia® in the morning, while one person took it in the evening. Ten precents of the subjects were at the age of between 30-40 years. Ten precents of the subjects were at the age of between 40-50 years and 80% were above 50 years.

The questionnaire included the following questions:
Age
Prior to VINIA, how often did you experience cold hands and feet? The grade was ranging from: rarely, sometimes, often, and always.
After VINIA, how often do you experience cold hands and feet? The grade was ranging from: rarely, sometimes, often and always.
Prior to VINIA, how would you rate the pain from your cold hands and feet? The grade was: no pain, mild pain, moderate pain, or intense pain.
After VINIA, how would you rate the pain from your cold hands and feet? The grade was: no pain, mild pain, moderate pain, or intense pain.
How many capsules do you take per day?
How long have you been taking VINIA?
What time of the day do you take VINIA?

These questions cover important aspects such as demographics (age), frequency and severity of symptoms, dosage, duration of usage, and timing of intake, providing valuable insights into the effects of VINIA on cold hands and feet symptoms.

### Results and Discussion:

### Prior to Vinia®

20% of the subjects often suffered from cold hands and/or feet.

40% of the subjects always suffered from cold hands and/or feet.

40% of the subjects sometimes suffered from cold hands and/or feet.

This breakdown shows the distribution of symptom severity among the subjects before they started taking Vinia®.

### After Vinia®

90% of the subjects asserted that they rarely suffer from cold hands and/or feet.

10% of the subjects asserted that they often suffer from cold hands and/or feet.

This indicates a significant improvement in symptom severity for the majority of subjects after taking Vinia®, with a large proportion reporting rare occurrences of cold hands and/or feet.

### Prior to Vinia®

The rate of pain was determined as follows:

40% asserted that they do not suffer from pain.

20% asserted that they suffer from mild pain.

30% asserted that they suffer from moderate pain.

10% asserted that they suffer from intense pain.

### After Vinia®

90% asserted that they do not suffer from pain.

10% asserted that they suffer from moderate pain.

This indicates a significant improvement in pain severity for most subjects after taking Vinia®, with a large proportion reporting no pain and only a very small percentage reporting moderate pain.

### CONCLUSIONS

For participants who suffered from cold hands and/or feet before consuming Vinia®, there was a significant improvement. Specifically, 90% of these participants reported rare occurrences of cold hands and/or feet after using Vinia®.

Similarly, for participants who suffered from pain before consuming Vinia®, there was significant improvement. Among them, 83% reported that they no longer feel any pain after the treatment with Vinia®.

### Example 2: Effect of Vinia® on Erectile Dysfunction

Vinia® has been on the market since 2018. The consumers which purchased the Vinia® product sent their feedback to the company asserting that Vinia® has beneficial effects on their erectile dysfunction symptoms.

Questionnaire were received from 8 subjects who had experienced a positive effect. The majority of the subjects except of one took one capsule of Vinia® daily. The last one took two capsules in the morning. All of the subjects took Vinia® for more than 3 months. All subjects took Vinia® in the morning. Five of the subjects were at the age of above 50 years, one was between 40-50 years and two were between 30-40 years.

The questionnaire included the following questions:
How would you rate your sexual performance prior to Vinia® in a scale of 1-5, wherein 5 is very satisfied and 1 is dissatisfied?
How would you rate your sexual performance after Vinia® in a scale of 1-5, wherein 5 is very satisfied and 1 is dissatisfied?
How many capsules do you take per day?
Do you take Vinia® prior to intercourse, if so, when do you take it?
How long have you been taking Vinia®?
What time of the day do you take Vinia®?

These questions aim to gather information about the subjects' sexual performance, dosage and timing of Vinia® intake in relation to sexual activity, duration of Vinia® usage, and timing of Vinia® intake throughout the day.

### Results and Discussion

The results are summarized in Table 1 below:

TABLE 1

| | | | How would you | How would you |
|---|---|---|---|---|
| | | | rate your sexual | rate your sexual |
| | | | performance prior | performance after |
| | | | to Vinia ®? | Vinia ®? |
| | | | A scale of 1-5 has | A scale of 1-5 has |
| | | How many | been used, | been used, |
| | Age | capsules a | wherein 5 is very | wherein 5 is very |
| Subject | range | day and | satisfied and 1 | satisfied and 1 |
| No. | (years) | timing | is dissatisfied. | is dissatisfied. |
| 1 | 50-60 | 1 capsule in | 3 | 3 |
| | | the morning | | |
| 2 | 60-70 | 1 capsule in | 3 | 4 |
| | | the morning | | |
| 3 | 60-70 | 1 capsule in | 2 | 3 |
| | | the morning | | |
| 4 | 30-40 | 1 capsule in | 4 | 5 |
| | | the morning | | |
| 5 | 40-50 | 1 capsule in | 4 | 5 |
| | | the morning | | |
| 6 | 30-40 | 1 capsule in | 3 | 5 |
| | | the morning | | |
| 7 | 60-70 | 1 capsule in | 3 | 4 |
| | | the morning | | |
| 8 | 60-70 | 2 capsules in | 2 | 3 |
| | | the morning | | |

The data suggest an improvement in sexual performance ratings for most participants after taking Vinia®. The ratings shifted towards higher satisfaction levels after Vinia® usage, indicating a positive effect on sexual performance.

### Conclusions

This conclusion underscores the effectiveness of Vinia® in addressing erectile dysfunction in the subjects studied. It suggests that Vinia® may offer significant benefits in improving sexual performance and erectile function.

It is to be understood that while the invention has been described in conjunction with the detailed description thereof the foregoing description is intended to illustrate and not limit the scope of the invention, which is defined by the scope of the appended claims.

Other aspects, advantages, and modifications are within the scope of the following claims.

1. A method of treating Raynaud's condition in a subject of need thereof comprising administering to the subject a composition comprising a therapeutically effective amount of red grape cell culture grown in vitro.

2. The method of claim 1, wherein the composition comprising the therapeutically effective amount of red grape cell culture grown in vitro further comprises a pharmaceutically or a dietary acceptable carrier.

3. The method of claim 1, wherein the Raynaud's condition is Raynaud's syndrome, Raynaud's phenomenon, and Raynaud's disease.

4. The method of claim 1, wherein the red grape cell culture grown in vitro are grown in bioreactors.

5. The method of claim 1, wherein the composition is in a form of a powder comprising a cell line culture of grape cells grown in vitro in a large scale up process, whereby the cell line culture of grape cells is derived from one or more of grape-berry cross section, grape-berry skin, grape-berry flesh, grape seed, grape embryo of seeded or seedless cultivars or grape seed coat; wherein the cell line culture of grape cells includes resveratrol in an amount of at least 1000 mg/kg powder.

6. The method of claim 1, wherein the composition is Vinia® or comprises Vinia®.

7. A method of treating a subject with erectile dysfunction and/or improving sexual performance, said method comprising the step of administering to said subject a composition comprising a therapeutically effective amount of red grape cell culture grown in vitro.

8. The method of claim 7, wherein the composition comprising a therapeutically effective amount of red grape cell culture grown in vitro further comprises a pharmaceutically acceptable carrier or a dietary acceptable carrier.

9. The method of claim 7, wherein the red grape cell culture grown in vitro are grown in bioreactors.

10. The method of claim 7, wherein the composition is in a form of a powder comprising a cell line culture of grape cells grown in vitro in a large scale up process, whereby the cell line culture of grape cells is derived from one or more of grape-berry cross section, grape-berry skin, grape-berry flesh, grape seed, grape embryo of seeded or seedless cultivars or grape seed coat; wherein the cell line culture of grape cells includes resveratrol in an amount of at least 1000 mg/kg powder.

11. The method of claim 7, wherein the composition is Vinia® or comprises Vinia®.

12.-18. (canceled)

19. (canceled)

20. A method of treating erectile dysfunction in a subject of need thereof comprising locally administering to the subject a topical composition comprising a therapeutically effective amount of red grape cell culture grown in vitro and a pharmaceutically or dermatologically acceptable carrier.

21.-22. (canceled)
